(19) United States
(12) Patent Application Publication
(10) Pub. No.: US 2006/0241759 A1
(43) Pub. Date: Oct. 26, 2006

(54) Oriented polymeric spinal implants

(75) Inventor: Hai H. Trieu, Cordova, TN (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: SDGI HOLDINGS, INC.

(21) Appl. No.: 11/113,190

(22) Filed: Apr. 25, 2005

(51) Int. Cl. A61F 2/44

(52) U.S. Cl. 623/17.11; 623/23.58; 264/331.11

(57) ABSTRACT

A polymeric spinal implant is disclosed wherein the polymer material is substantially uniformly oriented. The spinal implant is advantageous because the substantially uniformly oriented polymer material creates anisotropic properties, especially increased strength perpendicular to the orientation of the polymer material.

### FIELD OF THE INVENTION

Embodiments relate generally to artificial implants for use in orthopedics and other medical technologies, whereby the artificial implants are made of polymers injection molded in a manner to achieve oriented and anisotropic properties resulting in improved implant strength.

### BACKGROUND OF THE INVENTION

The intervertebral disc functions to stabilize the spine and to distribute forces between vertebral bodies. The intervertebral disc is composed primarily of three structures: the nucleus pulposus, the annulus fibrosis, and two vertebral end-plates. These components work together to absorb the shock, stress, and motion imparted to the human vertebrae. The nucleus pulposus is an amorphous hydrogel in the center of the intervertebral disc. The annulus fibrosis, which is composed of highly structured collagen fibers, maintains the nucleus pulposus within the center of the intervertebral disc. The vertebral end-plates, composed of hyalin cartilage, separate the disc from adjacent vertebral bodies and act as a transition zone between the hard vertebral bodies and the soft disc.

Intervertebral discs may be displaced or damaged due to trauma or disease. Disruption of the annulus fibrosis may allow the nucleus pulposus to protrude into the vertebral canal, a condition commonly referred to as a herniated or ruptured disc. The extruded nucleus pulposus may press on a spinal nerve, resulting in nerve damage, pain, numbness, muscle weakness, and paralysis. Intervertebral discs may also deteriorate due to the normal aging process. As a disc dehydrates and hardens, the disc space height will be reduced, leading to instability of the spine, decreased mobility, and pain.

One way to relieve the symptoms of these conditions is by surgical removal of a portion or all of the intervertebral disc. The removal of the damaged or unhealthy disc may allow the disc space to collapse, which would lead to instability of the spine, abnormal joint mechanics, nerve damage, and severe pain. Therefore, after removal of the disc, adjacent vertebrae are sometimes fused to preserve the disc space. Spinal fusion involves inflexibly connecting adjacent vertebrae through the use of bone grafts or metals rods. Because the fused adjacent vertebrae are prevented from moving relative to one another, the vertebrae no longer contact each other in the area of the damaged intervertebral disc and the likelihood of continued irritation is reduced. Spinal fusion, however, is disadvantageous because it restricts the patient's mobility by reducing the spine's flexibility, and it is a relatively invasive procedure.

Attempts to overcome these problems have led researchers to investigate the efficacy of implanting an artificial device to replace the damaged portion of the patient's intervertebral disc. One such prosthesis is an artificial nucleus implant for replacement of the nucleus pulposus. Nucleus implants are used when the nucleus pulposus of the intervertebral disc is damaged but the annulus fibrosis and vertebral end-plates are still sufficiently healthy to retain in the intervertebral disc. Nucleus replacement surgery involves removing the damaged nucleus pulposus of the intervertebral disc and insertion of the nucleus implant inside of the retained annulus fibrosis. The nucleus implant is often a molded bio-compatible polymer device designed to absorb the compressive forces placed on the intervertebral disc by adjacent vertebrae. For increased strength, the nucleus implant may be combined with an internal matrix of, for example, bio-compatible fibers. Some desirable attributes of a hypothetical nucleus implant include axial compressibility for shock absorbance, excellent durability to avoid future replacement, and bio-compatibility.

One example of a nucleus implant is disclosed in U.S. Pat. No. 6,620,196, incorporated herein by reference in its entirety, which discloses an intervertebral disc nucleus implant configurable in two positions: (i) a first straightened position for insertion through a small opening in the annulus; and (ii) a second folded position wherein the implant folds into a kidney shape similar to that of a natural nucleus pulposus. The implant is molded from a polymer and may have several different layers, including fiber jackets surrounding the elastic core for added rigidity.

Another example of a nucleus implant is disclosed in U.S. Pat. No. 6,264,695, incorporated herein by reference in its entirety, which discloses a nucleus implant with a two phase structure comprising a hydrophobic phase having high crystallinity and low water content and a hydrophilic phase having low crystallinity and high water content. The implant also has a negatively charged lubricious surface. The implant has an inherent shape, an insertion shape to which it may be deformed in order to facilitate insertion into the disk space, and an indwelling shape that the implant assumes after absorption of body fluids. Spherical, cylindrical, helixical, and ovate nucleus implant shapes are disclosed.

Another example of a nucleus implant is disclosed in U.S. Pat. No. 6,110,210, incorporated herein by reference in its entirety, which discloses a two-part implantable nucleus replacement. The two parts are joined together following insertion through the annulus into the evacuated nucleus to form a complete implant. The two parts are preferably fabricated from hydrogels that will expand to any given shape following implantation. In one illustrated embodiment, the implants are a tapered, angular shape like a three-dimensional trapezoid.

Yet another example of a nucleus implant is U.S. Pat. No. 6,387,130, incorporated herein by reference in its entirety, which discloses an implant that consists of a series of smaller implants fashioned to be inserted through a small opening in the annulus fibrosis. Each of the implants has a hole therethrough. A thin, elongated member passes through the hole and is used to guide the implants into place inside of the annulus. The implants resemble wedges with angled ends such that when they are pulled together inside of the annulus they form a single C-shaped implant.

Still another example of a nucleus implant is U.S. Pat. No. 5,976,186, incorporated herein by reference in its entirety, which discloses a hydrogel prosthetic nucleus. The prosthetic nucleus is in an elongated, rod-like shape that can be inserted through a small opening in the annulus. Inside of the annulus, the prosthesis coils into a spiral and expands to fill the evacuated volume inside of the annulus fibrosis.

Other molded polymeric implants requiring improved strength characteristics are known and used in the art. Polymeric implants could be used in for example, total joint replacements, such as hip replacement components (e.g., acetabular cup, cup inserts, femoral stems, etc.), knee replacement components, and shoulder replacement components. Polymeric implants also are useful in elbow implants, including the stem of the humeral and ulna components; in wrist implants, at the stem of the ulna component; and other known polymeric implants components. Molded and extruded polymeric implants, including oriented polymers are described in, for example, U.S. Pat. Nos. 5,679,299; 5,944,759; 6,692,497; 6,743,388; and 6,780,361, the disclosures of which are incorporated by reference herein in their entirety.

Spinal implants other than nucleus implants also benefit from the use of strengthened polymeric materials. These implants include plates, rods, screws, motion preserving disc replacement materials, facet arthroplasty devices, and other similar type materials. Typically, the spinal implants are comprised of biocompatible metal or metal composites due to the strength required of these implants. Polymeric implants having improved strength and load bearing characteristics would be desirable.

The description herein of problems and disadvantages of known apparatus, methods, and devices is not intended to limit the invention to the exclusion of these known entities. Indeed, embodiments of the invention may include one or more of the known apparatus, methods, and devices without suffering from the disadvantages and problems noted herein.

### SUMMARY OF THE INVENTION

An improved polymeric spinal implant would be advantageous. A number of advantages associated with the embodiments are readily evident to those skilled in the art, including economy of design and resources, ease of manufacture, cost savings, etc.

In accordance with these features, the embodiments provide a polymeric spinal implant device whereby the polymer is substantially uniformly oriented. The spinal implant can be made of any bio-compatible polymer and optional additives, and may be thermoplastic, semi-crystalline, liquid crystalline, thermosetting, amorphous, or any other appropriate type of bio-compatible polymer. In a preferred embodiment, the polymer chains are substantially uniformly oriented so that the implant has anisotropic properties.

In accordance with another embodiment of the invention, there is provided a method of making a spinal implant wherein the implant is molded from molten or semi-molten polymer. The molten or semi-molten polymer may be injected into a mold cavity to produce a substantially uniformly oriented polymer. The polymer is formed in a manner that encourages substantial uniform orientation of the polymer.

In accordance with another embodiment of the invention, an injection molding apparatus is provided that comprises at least one mold with a cavity, means for supplying molten or semi-molten polymer, and at least one communicating gate connecting the means for supplying the polymer and the mold cavity. The gate may be positioned with respect to the mold cavity to substantially uniformly orient the polymer chains during polymer solidification.

Still further features and advantages of the present invention are identified in the ensuing description.

### BRIEF DESCRIPTION OF THE DRAWINGS

FIG. 1 is an illustration of an exemplary spinal nucleus replacement implant showing placement of the gate and orientation of the polymer material.

FIG. 2 is an illustration of a number of spinal implants showing gate placement and polymer material orientation.

### DETAILED DESCRIPTION OF THE INVENTION

An improved artificial spinal implant would be advantageous. A number of advantages associated with the embodiments described herein are readily evident to those skilled in the art, including economy of design and resources, ease of use, cost savings, etc.

The following description is intended to convey a thorough understanding of the embodiments by providing a number of specifically preferred embodiments and details involving the manufacture of polymeric spinal implants having a substantially uniform orientation, whereby the implants have anisotropic properties and improved strength. Preferably, the polymeric spinal implants are made using an injection molding process. It is understood, however, that the embodiments are not limited to these specific embodiments and details, which are exemplary only. It further is understood that one possessing ordinary skill in the art, in light of known systems and methods, would appreciate the use of the embodiments of the invention for their intended purposes and benefits in any number of alternative embodiments, depending upon the specific design and other needs.

In one embodiment of the invention, there is provided a method for producing a spinal implant in a mold having at least one mold cavity. Molten or semi-molten polymer is injected into the mold cavity and allowed to cool and solidify. The manner in which the polymer is formed substantially uniformly orients the polymer chains and/or optional additives to provide a spinal implant having improved strength characteristics, when compared to an otherwise equivalent polymer formed using conventional injection molding techniques.

The spinal implant produced by the embodiments may be of any shape and size suitable for implantation. In one preferred embodiment, the spinal implant is a nucleus implant having a kidney-shaped disc designed to mimic the natural shape of the nucleus pulposus in the intervertebral disc. In another preferred embodiment, the spinal implant is a nucleus implant having an elongated body with a first end, a second end, and a central portion wherein the first end and second end are positioned in a folded, relaxed configuration adjacent to the central portion to form at least one inner fold. The preferred elastic body is deformable into a second, straightened, non-relaxed configuration for insertion through an opening in an intervertebral disc annulus fibrosis.

In another preferred embodiment, the spinal implant is a nucleus implant that comprises a plurality of implants, each with a hole passing therethrough. The implants have angled ends so that when the implants are positioned within the annulus fibrosis and pulled together, they form a C-shaped implant. In another preferred embodiment, the spinal implant is deformable such that it can be molded into an inherent shape, deformed into an insertion shape, and attain an indwelling shape upon implantation. The molded shape may be spherical, cylindrical, helixical, ovate, or any other appropriate shape. In another preferred embodiment, the spinal implant is a two part implant that is joined together following insertion into the evacuated nucleus pulposus. The implant may have an angular, tapered shape like a three-dimensional trapezoid. In another preferred embodiment, the implant is molded into an elongated rod-like shape. Upon insertion into the evacuated nucleus pulposus, the rod coils into a spiral shape.

The preferred nucleus implant produced by the methods described herein may be only a part or layer of a multi-part or multi-layered implant. For example, the nucleus implant may be the center of a nucleus implant surrounded by a fabric or another polymer layer. The elastic center may take on any of the shapes discussed herein or any other appropriate shape for implantation. In another preferred embodiment, the spinal implant produced by this method is the center layer of a three-layered nucleus implant. One possessing ordinary skill in the art, in light of known systems and methods, will appreciate the myriad implant configurations that may be produced by this method.

The spinal implant in accordance with other embodiments is any one of the known fixation devices and components. Such implants include fixation plates positionable over two, three, or more adjacent vertebral bodies. Other suitable implants includes rods positioned through multi-axial screws or other pedicle screw devices, fusion cages, tethers attached to bone anchors on separate vertebral bodies, bone anchors, claws, hooks, facet arthroplasty devices, articulating surfaces, translaminar screws, and other known implants suitable for implantation in a patient's vertebral column. Such implants typically require enhanced strength at least in one direction to stabilize the spine, and to provide enhanced load bearing capabilities. Polymeric implants whose polymer chains are oriented in substantially one direction are particularly suitable for use as spinal implants requiring improved strength characteristics.

The mold used to fabricate the spinal implants may be made of any suitable material. For example, the mold may be made of a metal such as aluminum, steel, iron, and mixtures thereof. Alternatively, the mold may be made of a ceramic. The mold may be cooled, for example by a refrigerated liquid or air, in order to promote fast crystallization of the molten or semi-molten polymer following injection into the mold cavity. Alternatively, the mold may be heated so as to impede the crystallization of the molten or semi-molten polymer following injection into the mold cavity in order to promote slower, more perfect crystal formation. The mold may also have multiple cavities. One possessing ordinary skill in the art, in light of known systems and methods, will appreciate the myriad configurations that the mold may take.

In molded polymeric materials, increased anisotropic rigidity or strength may be achieved by substantially orienting the polymer chains in the material during processing. Polymers are large, long chains of organic molecules. By careful processing, for example slow cooling from melt state or application of pressure, polymeric materials may be produced with a continuous, directionally oriented crystalline structure throughout the material. Alternatively, or in addition, an optionally amorphous or otherwise less crystalline polymer (or preferably a highly crystalline polymer) is combined with an additive that can be substantially oriented in one direction to provide increased anisotropic rigidity or strength to the ultimately formed material. A crystalline polymer (or oriented amorphous polymer, or polymer with oriented reinforcement additives) typically has increased strength when measured perpendicular to the orientation of the polymer chains, similar to the increased rigidity of wood measured perpendicular to the orientation of its grain. Injection molding of plastic articles, wherein molten or semi-molten polymer is injected under pressure into molds of the article to be produced, is known to be capable of producing directionally-oriented plastics because of the high shear forces within the molten or semi-molten polymer which results in flow-induced orientation during injection.

Any method can be used to substantially orient the polymeric material. Throughout this description, the expression “substantial orientation,” as it refers to the polymeric material (e.g., the polymer chains, the reinforcing additive, or both), denotes orientation in substantially one direction such that the majority of materials essentially have the same orientation. That is, more than 20% of the oriented materials have the same orientation, or are positioned in the same direction. Preferably, more than 40% of the oriented materials have the same orientation, and more preferably, more than 60%, most preferably 80% and even more preferably 90% of the oriented materials have the same orientation. Having the same orientation typically means the materials face or are positioned in substantially the same direction, plus or minus about 30 degrees, preferably, plus or minus 20 degrees, and most preferably, plus or minus 10 degrees.

The substantially uniformly oriented polymer chains may be oriented in many different configurations. As described previously, the “uniformly oriented” polymeric material has a portion that is oriented in substantially the same direction. This does not mean that the direction must be linear. For example, the oriented materials may be present in an organized, relatively non-random pattern, such as in a series of parallel lines, or a series of aligned curves, or a series of concentric arcs. In some embodiments, the substantially uniformly oriented polymer material is oriented radially about a center. The preceding embodiments are examples only of the numerous different configurations that the substantially uniformly oriented polymer material may assume when viewed as a planar cross-section of the implant.

Because the substantially uniformly oriented polymer material provides extra rigidity when measured perpendicular to the plane in which the polymer material is oriented, the spinal implant may be considered self-reinforced when just a polymer material is used, as opposed to other polymer implants in other embodiments that use fibers or other additives to impart increased strength to the implant. In preferred embodiments of the present invention, the plane in which the polymer material is substantially uniformly oriented is perpendicular to the compressive force to which the spinal implant will be subjected.

One preferred method used to substantially orient the polymeric material includes modifying the manufacturing process to orient the material, such as modifying the position of the gate used in the injection molding process, modifying the solidification parameters (i.e., cooling temperature and time), and/or modifying the injection conditions (i.e., flow rate, temperature, viscosity, etc.). Another method capable of substantially orienting the polymeric materials includes the use of reinforcing additives that themselves orient during the manufacturing (e.g., by virtue of the flow direction or polymeric cooling process, or by use of magnetic or electrically or optically active materials that can be oriented during manufacture by application of external magnetic, electrical, or optical energy, etc.). An additional method of substantially orienting the polymeric materials includes post molding processes, such as stretching, compression, hot isostatic pressing, twisting, etc., that can serve to orient the polymer chains and/or optional additives.

In one embodiment, the polymeric material is substantially oriented by modifying the position of the communicating gate used in the injection molding process. The position of the communicating gate may be any position such that the polymer material entering the mold cavity (polymer and/or optional additives) will be substantially uniformly oriented during polymer solidification or crystallization. While not intending on being bound by any theory of operation, the location of the gate relative to the mold cavity during injection molding is thought to be important in controlling the orientation of the polymer chains because it determines in part if a smooth, even flow of molten or semi-molten polymer will fill the implant or if the flow will be turbulent and uneven. It is believed that orienting the polymer chains in a plastic article yields an article that has anisotropic properties (i.e. properties that are dependent upon the direction of measurement). In a plastic article, increased strength is observed when measured relatively perpendicularly to the oriented polymer chains. In a preferred embodiment, the communicating gate is situated so as to cause the polymer chains to orient perpendicular to the compressive loads that the spinal implant will be expected to endure after implantation. In another preferred embodiment, the communicating gate is located in the center of the mold cavity. One skilled in the art will appreciate that the gate location desired to produce a substantially uniformly oriented polymer material will vary depending upon the shape of the implant to be molded.

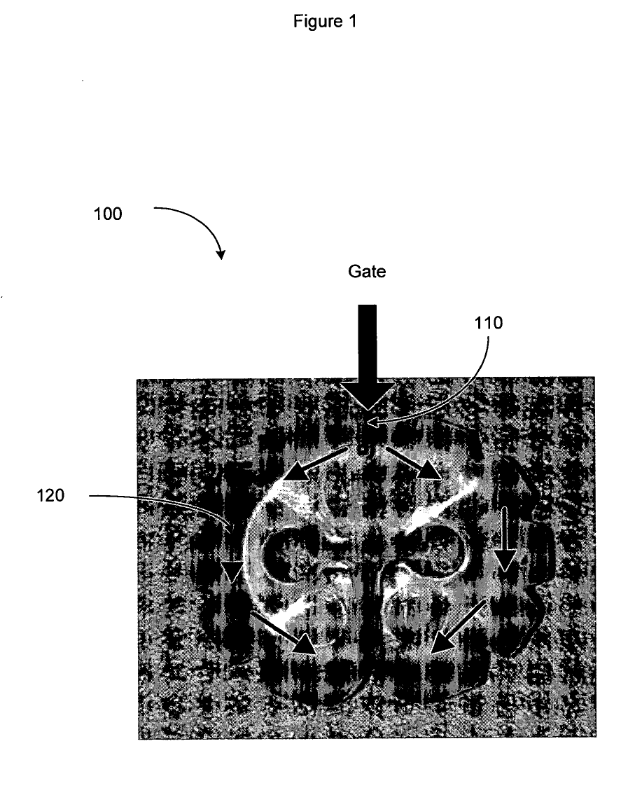

The figures appended hereto illustrate preferred embodiments showing gate placement and polymer material orientation. FIG. 1 illustrates an exemplary nucleus replacement device 100 whereby the gate 110 is located substantially in the center of the mold, and along the thickness of the nucleus replacement device 110, as opposed to above or below the device. As shown in FIG. 1, the polymer material wills have a substantially uniform orientation due to the material flow of the polymer material as it enters the mold through the gate. The substantially uniform polymer material orientation is depicted by arrows 120. A skilled artisan will appreciate that placing the gate on the top or bottom of the nucleus replacement device 100 would not result in the same substantially uniform polymer orientation. The substantially uniform polymer material orientation shown in FIG. 1 will improve that material properties and hoop stress resistance of the device 100, which will help prevent the device from spreading open after insertion.

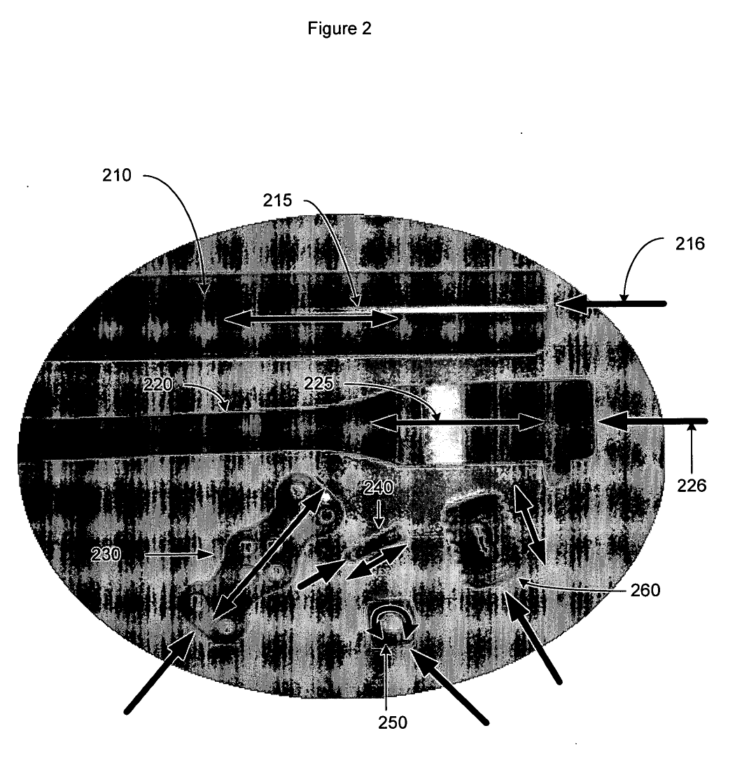

FIG. 2 illustrates a number of spinal implants, and how the gate placement can influence polymer material orientation to provide an implant having improved properties. For example, a spinal rod 210 can be formed by placing the gate 216 axially at one end and injecting the polymer material longitudinally along the rod, which will result in polymer orientation along the lines 215. Similarly, bone plate 220 can be formed by placing gate 226 at one end and injecting the polymer material longitudinally along the plate to provide polymer orientation substantially along the lines 225. The arrows for the remaining implants relate to the same features as described in the spinal rod 210 and bone plate 220: (i) a single arrow indicating gate placement; and (ii) a double arrow indicating polymer material orientation. FIG. 2 further illustrates the polymer material orientation for a molded anterior cervical plate 230, a molded screw 240, a molded cervical cage 250, and a molded lumbar cage 260. Using the guidelines provided herein, those skilled in the art will be capable of fabricating any of a variety of spinal implants to provide the desired polymer material orientation.

Another method of substantially orienting the polymer material includes the use of reinforcing additives that themselves can be oriented during manufacture of the implant. In this manner, a less crystalline polymer material can be used, even amorphous polymeric materials. Reinforcing additives that are suitable for use in these embodiments include those that can be oriented, either naturally during the implant manufacturing process (e.g., by virtue of the linear flow of the polymer material), or that can be oriented by application of external energy, such a electricity, heat, magnetism, light, radiation, etc. For example, fibers can be used that are short and have a relatively small aspect ratio whereby the fibers are oriented in a direction of polymer flow by virtue of their rod-like shape. Particles having a high aspect ratio also can be used with higher flow rates and higher viscosity polymer compositions. Blends of high and low aspect ratio fibers also may be used.

Other suitable reinforcing additives include fibers or other materials that can be oriented by application of external energy. Magnetic fibers can be used and the polarity of the material in the mold can be changed to effect orientation of the fibers. Light sensitive polymers, cross-linking agents, or optically active (chirally active) materials can be used, and then the polymer material subjected to a given wavelength of light to orient the polymer material. Two different types of polymers may be used, whereby the polymers have different crystallinity or orientability. This embodiment would permit the use of amorphous polymers. Suitable reinforcing additives for use in the embodiments include, for example, metallic fibers, ceramic fibers, polymeric fibers, carbon fibers, KEVLAR® fibers, SPECTRA® fibers, polyester fibers, hydroxyapatite particles, short fibers, long fibers, continuous fibers, woven or spun bonded fibers, filaments, and the like.

An additional method of substantially orienting the polymer material includes that addition of 3-dimensional materials to the polymer material. Suitable 3-dimensional materials include mesh structures, woven or braided, that can facilitate orientation of the polymer material either during solidification or during a post molding process such as stretching.

Beneficial post-molding operations may be performed on the spinal implant. In a preferred embodiment, the spinal implant may be annealed at temperatures below the melting point of the polymer. The annealing process permits the polymer chains on the outside faces of the implant to re-crystallize and align themselves with the polymer chains in the rest of the implant body. This is beneficial because the crystalline structure of the polymer chains on the outside faces of the implant may contain imperfections due to the rapid cooling of the molten or semi-molten polymer upon contacting the surfaces of the mold cavity during injection.

Other post-molding operations include stretching, compression, isostatic pressing, twisting, annealing, freezing, heating, forging, treatment with light and other forms of irradiation, thermo-mechanical light or radiation energy to manipulate the matrix, etc. It is known to stretch polymers shortly after forming them, while still not fully cooled, and substantially orient the polymer chains, even for amorphous polymeric materials such as polymethylemethacrylate (PMMA), polycarbonates, and polysulfone polymers. Suitable drawing processes are described in, for example, U.S. Pat. Nos. 4,963,151, 4,735,625, 5,037,442, 4,895,573, 3,992,725, 4,718,910, 4,851,004, 5,080,680, 5,180,395, 5,197,990, 4,743,257, 5,171,288, 5,135,804, 4,737,012, 4,403,012, 4,961,647, 5,415,474, and 5,679,299, the disclosures of which are incorporated by reference herein in their entirety.

While injection molding is a preferred method of making the spinal implant, skilled artisans will recognize that the polymer implant may be produced by other molding processes. Suitable processes for fabricating a spinal implant wherein the polymer material is substantially uniformly oriented include compression molding, transfer, cutting, dipping, coating, extrusion, protrusion, and insert molding. Polymeric spinal implants may be manufactured using any of these processes.

In another embodiment of the invention, there is provided a spinal implant comprising a polymer material wherein the polymer material is substantially uniformly oriented. The expression “polymer material” denotes the native polymer itself, or a polymer composition comprising additives, other polymers, or macromolecular composites. The material itself is oriented meaning that the polymer chains may be oriented, or the additives are oriented, or in the case of a macromolecular composite, portions of the composite are oriented.

The spinal implant may be in any appropriate shape for implantation, or in the case of a nucleus replacement or fusion device, in an appropriate shape to replace the nucleus pulposus of the intervertebral disc. As discussed above, these shapes include deformable rods, a kidney-shaped prosthesis, spherical, cylindrical, helixical, ovate, trapezoidial, spiral, screw shape, rectangular plate shaped, and any other appropriate shape or configuration. In a preferred embodiment, the polymer material is substantially uniformly oriented perpendicular to the compressive load of the vertebral column on the spinal implant.

Any polymer may be used in the invention so long as it is capable of forming a suitable spinal implant, and the polymer material is capable of being shaped by a suitable shaping process. To be suitable for use as a spinal implant, the polymer may preferably have sufficient mechanical stability to absorb the compressive shock placed on the intervertebral disc by the adjacent vertebrae. Additionally, the polymer should be bio-compatible. In a preferred embodiment, mixtures of appropriate polymers may be used.

Examples of suitable biocompatible polymeric materials include elastic materials such as elastomeric materials, hydrogels, thermoplastic polymers, liquid monomers, polymer dispersions, gel based polymers, liquid crystal polymers, macromolecular composites, crystalline polymers, semi-crystalline polymers, amorphous polymers, other hydrophilic polymers, and composites thereof. Suitable elastomers include silicone, polyurethane, copolymers of silicone and polyurethane, polyolefins such as polyisobutylene and polyisoprene, neoprene, nitrile, vulcanized rubber, and combinations thereof. Suitable hydrogels include natural hydrogels, and those formed from polyvinyl alcohol, acrylamides such as polyacrylic acid and poly(acrylonitrile-acrylic acid), polyurethanes, polyethylene glycol, poly(N-vinyl-2-pyrrolidone), polyacrylates such as poly(2-hydroxy ethyl methacrylate) and copolymers of acrylates with N-vinyl pyrrolidone, N-vinyl lactams, acrylamide, polyurethanes and polyacrylonitrile, and other similar materials that form a hydrogel. The hydrogel materials may further be cross-linked to provide further strength to the implant. Examples of polyurethanes include thermoplastic polyurethanes, aliphatic polyurethanes, segmented polyurethanes, hydrophilic polyurethanes, polyether-urethane, polycarbonate-urethane and silicone polyetherurethane. Other suitable hydrophilic polymers include naturally-occurring materials such as glucomannan gel, hyaluronic acid, polysaccharides, such as cross-linked carboxyl-containing polysaccharides, and combinations thereof. Other bio-compatible polymers which have a sufficient mechanical stability include thermoplastic materials such as polyesters, polyamides, polyethylene terephtalate, high-density polyethylene, polypropylene, polysulfones, polyphenylene oxides, polyetheretherketone and the like. Other polymers include the following bioresrobable materials: polylactide, polyglycolide, poly(lactide-co-glycolide), poly(dioxanone), poly([epsilon]-caprolactone), poly(hydroxylbutyrate), poly(hydroxylvalerate), tyrosine-based polycarbonate, polypropylene fumarate, and combinations thereof. In preferred embodiments, a tensile strength of at least about 1 Mpa is desired, although tensile strengths in the range of about 10 Mpa to about 250 Mpa are more preferred, more preferably in the range of from about 20 Mpa to about 150 Mpa.

In another preferred embodiment, beneficial additives may be added to polymer. These beneficial additives may include antibiotics, anti-retroviral drugs, nutrients, preservatives, binders, and any other bio-compatible additive. Osteoconductive or osteoinductive agents also may be added to the polymer implant during its manufacture, or coated thereon to encourage osseointegration between adjacent bony tissue and the spinal implant, if such osseointegration is desirable. Skilled artisans will recognize other suitable additives, and any additives now known or later discovered may be used in the context of the embodiments described herein.

Another embodiment includes an apparatus for producing spinal implants. The apparatus includes at least one mold with a cavity therein, means for supplying a molten or semi-molten polymer, and at least one communicating gate connecting the means for supplying the polymer and the mold cavity wherein the gate is positioned with respect to the mold cavity to substantially uniformly orient the polymer material during polymer solidification. One skilled in the art will appreciate that there are myriad means for supplying a molten or semi-molten polymer to the gate and mold cavity.

An example of a typical means for supplying molten or semi-molten polymer has a hopper wherein polymeric material is fed. The hopper places polymeric material (and optional additives) on a feed screw. The polymeric material is fed by the feed screw through a heating cylinder to melt the polymer. The feed screw is rotated by a screw motor via a coupling. The feed screw also functions as a hydraulic ram that is reciprocally moved back and forth in the hydraulic cylinder when a predetermined amount of material, as detected by the pressure within the cylinder, accumulates in front of the screw. The molten or semi-molten polymer then is forced through the gate and into the mold cavity where it is held, under pressure, until it solidifies. The mold is then opened, the part removed and the process repeated. Optionally, the mold is opened prior to solidification of the polymer material, and the material stretched to orient the polymer material. The mold cavity can be duplicated at several locations in the mold such that multiple parts can be produced simultaneously.

The apparatus mold may, as described above, be made of any metal, alloy, other mixtures of metals, ceramics, cements, or any other suitable material. The polymer may, as described above, be any bio-compatible polymer capable of being molded and possessing sufficient mechanical stability to absorb the compressive shock placed on the intervertebral disc by the adjacent vertebrae. Examples of such polymers include, but are not limited to, polyesters, polyamides, polyethylene terephtalate, high-density polyethylene, polypropylene, polysulfones, polyphenylene oxides, polyetheretherketone, silicone, polyurethane, copolymers of silicone and polyurethane, polyolefins, such as polyisobutylene and polyisoprene, neoprene, nitrile, vulcanized rubber, and combinations thereof.

In accordance with one preferred embodiment of the invention, the location of the gate is controlled such that the polymer material, when solidified, is substantially uniformly oriented. Depending on the shape of the mold cavity, the location of the gate will vary, as will be appreciated by those skilled in the art. For example, for a generally cylindrically-shaped mold (e.g., cylindrical disc), the gate typically is placed at or near the center of the circular cross-section of the cylinder. For a kidney-shaped mold cavity, or a “C”-shaped mold cavity, as is typically employed in forming a spinal nucleus implant, the gate is positioned at or near the geometric center of the mold cavity. Using the guidelines provided herein, those skilled in the art will be capable of positioning the gate to substantially uniformly orient the polymer during its crystallization.

The invention now will be explained by reference to the following non-limiting examples.

### EXAMPLE 1

PURASIL® 20 80A silicone polyether urethane (The Polymer Technology Group, Berkeley, Calif.) was injection molded in a “C”-shaped mold cavity with the gate placed in the center of the cavity to form a spinal nucleus implant. The gate placement, the molding conditions, and the resulting material flow together induced molecular orientation along the curved C-shape of implant. This partial orientation was found to strengthen the device. The partial molecular orientation was reduced upon thermal treatments, which lead to reduction in mechanical properties.

### EXAMPLE 2

BIONATE® 80A polycarbonate urethane (The Polymer Technology Group, Berkeley, Calif.) is injection molded in the same “C”-shaped mold cavity as described in example 1, with the gate placed in the center of the cavity. The gate placement, the molding conditions, and the resulting material flow together induce molecular orientation along the curved C-shape of implant. This partial orientation was found to strengthen the device.

### EXAMPLE 3

CARBOSIL® 20 80A silicone polycarbonate urethane (The Polymer Technology Group, Berkeley, Calif.) is injection molded in the same “C”-shaped mold cavity as described in example 1, with the gate placed in the center of the cavity. The gate placement, the molding conditions, and the resulting material flow together induce molecular orientation along the curved C-shape of implant. This partial orientation was found to strengthen the device.

### EXAMPLE 4

ELASTEON® 3 silicone polyurethane (Aortech, UK) is injection molded in the same “C”-shaped mold cavity as described in example 1, with the gate placed in the center of the cavity. The gate placement, the molding conditions, and the resulting material flow together induce molecular orientation along the curved C-shape of implant. This partial orientation was found to strengthen the device.

The invention has been described with reference to the non-limiting examples and particularly preferred embodiments. Those skilled in the art will appreciate that various modifications may be made to the invention without departing significantly from the spirit and scope thereof.

What is claimed is:

1. A method for producing a spinal implant comprising a substantially uniformly oriented polymer material, comprising:
providing a polymer material suitable for molding into a spinal implant;
supplying the polymer material to the mold;
orienting the polymer material to form a substantially uniformly oriented polymer material;
molding the polymer material into a spinal implant; and
allowing the polymer to solidify.

2. The method of claim 1, wherein molding the polymer material takes place prior to orienting the polymer material.

3. The method of claim 1, wherein molding the polymer material takes place after orienting the polymer material.

4. The method of claim 1, wherein the polymer material comprises a polymer and a reinforcing additive.

5. The method of claim 4, wherein the reinforcing additive is selected from the group consisting of metallic fibers, ceramic fibers, polymeric fibers, carbon fibers, KEVLAR® fibers, SPECTRA® fibers, polyester fibers, hydroxyapatite particles, short fibers, long fibers, continuous fibers, woven or spun bonded fibers, filaments, and mixtures thereof.

6. The method of claim 4, wherein the reinforcing additive is capable of orientation by application of external energy.

7. The method of claim 6, further comprising supplying external energy to the polymeric material to substantially uniformly orient the reinforcing additive.

8. The method of claim 7, wherein the external energy is selected from the group consisting of heat, light, magnetism, electrical, mechanical and irradiation.

9. The method of claim 1, wherein supplying the polymer material to the mold comprises supplying the polymer material through a communicating gate.

10. The method of claim 9, wherein the communicating gate is positioned so as to cause the polymer material to substantially uniformly orient perpendicular to the compressive load to which the spinal implant will be subjected.

11. The method of claim 10, wherein the communicating gate is positioned in the center of the mold.

12. The method of claim 10, wherein the communicating gate is positioned at one end of an elongated mold having a length greater than its effective diameter, thereby providing a polymer material substantially uniformally oriented along the length of the mold.

13. The method of claim 1, wherein orienting the polymer material comprises supplying the polymer material to the mold in a manner that the polymer material is substantially oriented in a direction substantially parallel to the supplying direction.

14. The method of claim 1, wherein orienting the polymer material comprises further processing the polymer material after it has been molded.

15. The method of claim 14, wherein further processing comprises stretching.

16. A polymeric spinal implant comprising a substantially uniformly oriented polymer material.

17. A polymeric spinal implant comprising a substantially uniformly oriented polymer material, prepared by the method of claim 1.

18. The implant of claim 17, wherein the polymer material is substantially uniformly oriented perpendicular to the compressive load to which the implant will be subjected.

19. The implant of claim 17, wherein the polymer material comprises a polymer selected from the group consisting of elastomeric materials, hydrogels, thermoplastic polymers, liquid monomers, polymer dispersions, gel based polymers, liquid crystal polymers, macromolecular composites, crystalline polymers, semi-crystalline polymers, amorphous polymers, hydrophilic polymers, and composites or mixtures thereof.

20. The implant of claim 19, wherein the polymer is selected from the group consisting of silicone, polyurethanes, copolymers of silicone and polyurethane, polyisobutylene, polyisoprene, neoprene, nitrile, vulcanized rubber, natural hydrogels, hydrogels formed from polyvinyl alcohol, polyacrylic acid, poly(acrylonitrile-acrylic acid), polyethylene glycol, poly(N-vinyl-2-pyrrolidone), poly(2-hydroxy ethyl methacrylate), copolymers of acrylates with N-vinyl pyrrolidone, N-vinyl lactams, acrylamide, polyacrylonitrile, thermoplastic polyurethanes, aliphatic polyurethanes, segmented polyurethanes, hydrophilic polyurethanes, polyether-urethane, polycarbonate-urethane, silicone polyetherurethane, glucomannan gel, hyaluronic acid, cross-linked carboxyl-containing polysaccharides, polyesters, polyamides, polyethylene terephtalate, high-density polyethylene, polypropylene, polysulfones, polyphenylene oxides, polymethylmethacrylate, polyetheretherketone, polylactide, polyglycolide, poly(lactide-co-glycolide), poly(dioxanone), poly([epsilon]-caprolactone), poly(hydroxylbutyrate), poly(hydroxylvalerate), tyrosine-based polycarbonate, polypropylene fumarate, and mixtures and combinations thereof.

21. The implant of claim 17, wherein the polymer material comprises a reinforcing additive.

22. The implant of claim 21, wherein the reinforcing additive is selected from the group consisting of metallic fibers, ceramic fibers, polymeric fibers, carbon fibers, KEVLAR® fibers, SPECTRA® fibers, polyester fibers, hydroxyapatite particles, short fibers, long fibers, continuous fibers, woven or spun bonded fibers, filaments, and mixtures thereof.

23. The implant of claim 17, wherein the polymer material further comprises an additive selected from the group consisting of antibiotics, anti-retroviral drugs, nutrients, preservatives, binders, osteoconductive agents, osteoinductive agents, and mixtures thereof.

24. The implant of claim 17, wherein the implant has a tensile strength within the range of about 10 Mpa to about 250 Mpa.

25. The implant of claim 24, wherein the implant has a tensile strength within the range of from about 20 Mpa to about 150 Mpa.
